(19) United States
(12) Patent Application Publication
(10) Pub. No.: US 2019/0015385 A1
(43) Pub. Date: Jan. 17, 2019

(54) VITAMIN E PHOSPHATE OR ACETATE FOR USE IN THE TREATMENT AND PREVENTION OF BIOFILM INFECTIONS

(71) Applicants: EON MEDICA S.R.L., Monza (IT); FLAME S.R.L., Masate (IT); BEST PATIENT LIFE S.R.L., Milano (IT)

(72) Inventors: Tommaso BRENICCI, Monza (IT); Maria Emanuela CECCA, Milano (IT)

(21) Appl. No.: 16/068,576

(22) PCT Filed: Jan. 5, 2017
(86) PCT No.: PCT/IT2017/000001

§ 371 Date: Jul. 6, 2018

(30) Foreign Application Priority Data
Jan. 8, 2016 (IT) 102016000001232

(51) Int. Cl. A61K 31/355; A61K 31/665; A61K 31/5415; A61K 9/00; A61K 47/36; A61L 27/34; A61L 27/54; A61L 29/08; A61L 29/16; A61L 24/00; A61L 24/08; A01N 43/16

(52) U.S. Cl. CPC A61K 31/355; A61K 31/665; A61K 31/5415; A61K 9/0014; A61K 47/36; A01N 43/16; A61L 27/54; A61L 29/085; A61L 29/16; A61L 24/0015; A61L 24/08; A61L 27/34

(57) ABSTRACT

The present invention concerns vitamin E selected among vitamin E phosphate, optionally in combination with methylene blue, and/or vitamin E acetate in combination with methylene blue for use in the treatment and prevention of biofilm infections or as an antifungal agent by means of the application of vitamin E as defined above or in combination with a biocompatible vector on inert or living surfaces.

The present invention concerns Vitamin E for use in the treatment and prevention of biofilm infections.

More specifically, the invention concerns vitamin E selected from among vitamin phosphate, optionally in combination with methylene blue, and/or vitamin E such as vitamin E acetate, in combination with methylene blue for use in the treatment and prevention of biofilm infections or as an antifungal agent by means of the application of vitamin E as defined above or in combination with a biocompatible vector on inert or living surfaces.

Bacterial and biofilm infections due to implanted or injected biomaterials still represent a serious complication in surgery such as in orthopaedic surgery, trauma surgery, cardiovascular, urological, and dental surgery for example.

Acute and chronic infections can develop in many cases of prosthetic implants. In cases in which an inert foreign body is implanted in tissue that is already damaged or weakened, competition develops between the bacteria and the cells of the immune system for the colonization of the implant surface. However, with respect to the cells of the immune system, the bacteria have the advantage of having faster reproductive processes and exceptional flexibility in adapting to the environment.

Biofilm infections, such as infections associated with implants and catheters, or infections that cause pneumonia in patients suffering from cystic fibrosis, chronic wounds, or chronic otitis media, affect millions of people throughout the world each year and cause many deaths.

Bacteria generally exhibit two forms of life during their growth and proliferation. In one form, the bacteria appear as single independent cells (planktonic form), whereas in the other form, the bacteria are organized into sessile aggregates. This latter form is commonly indicated as a phenotype for growth in biofilms.

Chronic infections caused by biofilms are characterized by high resistance to antibiotics and to many other conventional antimicrobial agents, and by a strong ability to elude the defences of the host.

It is therefore common practice to adopt local antibiotic treatment as an alternative or in addition to systemic antibiotic treatment, for the prophylaxis and prevention of bacterial infections originating from implanted biomaterials. In fact, it is important to treat infection locally so as to obtain a high concentration of the active ingredient in the site of the infection, thereby avoiding systemic toxicity, and to achieve a more thorough eradication of the infection. Unfortunately, however, treatment of a bacterial infection with an antibiotic or antimicrobial agent does not always guarantee eradication of the biofilm, when the latter is present.

There are various known coating biomaterials or “drug delivery systems” which contain antibiotics that are used locally as coatings for prostheses. For example, some materials are disclosed in patent applications US 2004/0013626 A1, WO 2005/032417, EP 1666518 A1, WO2013119582 A1, and WO2010086421 A1 or in the following articles: “New amphiphilic lactic acid oligomer-hyaluronan conjugates: synthesis and physicochemical characterization”, Pravata L. et al, Biomacromolecules (2008) 9, 340-348; “New graft copolymers of hyaluronic acid and polylactic acid: synthesis and characterization”, Palumbo F. S. et al, Carbohydrate Polymers (2006) 66, 379-385; “Synthesis of novel graft copolymers of hyaluronan, polyethyleneglycol and polylactic acid”, Pitarresi G. et al, Macromolecules an Indian Journal, Vol. 3, Issue 2, August 2007, 53-56; “Self-setting hydroxyapatite cement: A novel skeletal drug-delivery system for antibiotics” Duncan Yu et al., (1992), 81, 529-531. However, the coating biomaterials have various drawbacks. In the first place, the antibiotics contained in the biomaterial are of limited duration, to the extent that they may not be completely efficient at the time of implantation. A second drawback of known coating biomaterials is that they do not allow for adaptation to specific needs. For example, they do not permit the antibiotic to be changed in the case of a patient's known intolerance to a given antibiotic or in the case in which a particular antibiotic, differing from the one already present in the biomaterial, must be adopted. Moreover, known coating biomaterials do not allow for changing the dosage of the antibiotic according to a specific need. Lastly, given that known coating biomaterials contain antibiotics, they may not be effective owing to phenomena relating to resistance to these types of drugs.

In fact, biofilm formation represents a serious threat owing to the antimicrobial resistance (AMR) of the biofilm itself and it is therefore the focus of ever-increasing interest in various countries and in various fields of research. The governments of countries throughout the world are beginning to focus attention on this very serious problem and many researchers are conducting studies aimed at finding new methods for treating surgical site infections that do not include the use of antibiotics.

Vitamin E is a fat-soluble antioxidant and anti-inflammatory compound that is used in many cosmetic products. The term vitamin E indicates a group of tocopherols and tocotrienols, of which α-tocopherol has the highest biological activity. α-tocopheryl acetate (vitamin E acetate) is the acetic ester of α-tocopherol, a highly hydrophobic viscous oil that has greater stability compared to the unreduced form.

When applied locally, vitamin E deactivates the unstable free radicals, supplying one of its electrons to the free radical missing an electron, making it more stable. As a result, Vitamin E protects tissue from harmful effects resulting from exposure to exogenous toxic agents such as pollutants, chemical products and sunlight, preventing the propagation of free radicals.

The European Food Safety Authority (EFSA) has confirmed that the consumption of Vitamin E contributes to the protection of the cellular constituents from oxidative damage with evident benefits in terms of health.

Vitamin E is considered to be safe for chronic use, even at doses of up to 1000 mg per day. Many long-term studies conducted on humans with much higher doses of vitamin E report no negative effects. The LD50 or the toxic dose required to kill 50% of rats or mice is equal to 4000 mg of vitamin E/kg of a rat or mouse.

Along with other biological functions, vitamin E has proved to be capable of improving specific aspects of the immune response which seem to decrease in tissue damage or infections. A clinical study conducted on elderly subjects residing in a nursing home reports that daily administration of vitamin E lowered the risk of infections of the upper respiratory tract, particularly the common cold, but that it did not have an effect on infections of the lower respiratory passages.

Therefore, based on the above considerations, vitamin E can be advantageously used even at very high doses, without the risk of side effects.

Preliminary results have recently been published on the effects of vitamin E against pathogenic bacteria such as Staphylococcus aureus, Staphylococcus epidermidis, Escherichia coli, Proteus spp., Klebsiella spp., Pseudomonas aeruginosa and Enterobacter spp. (Journal of Biotechnology Research Center, Vol. 7 no. 2, 2013).

For example vitamin E is known to be used in combination with ultra-high molecular weight polyethylene (UHMWPE) to reduce microbial adhesion on surfaces of biomaterials used in orthopaedics (Banche G. et al, Clinical Orthopaedics and Related Research, Springer, New York LLC, Vol. 473, no. 3, 2014, pp. 974-986; Banche et al., The Bone & Joint Journal, 2014, Vol. 96-B, no. 4, pp. 497-501). Moreover, bacterial adhesion and biofilm accumulation have been found to decrease on surfaces made of polylactic acid and vitamin E (Davide Campoccia et al., Journal of Biomedical Materials Research, Part A, Vol. 103, no. 4, 2014, pp. 1447-1458).

In light of the considerations stated hereinabove, there is an evident need for new anti-biofilm formulations that are capable of destroying or inhibiting biofilms and that are also safe for human tissues.

The solution according to the present invention lies within this context, as it is intended to offer new formulations that are more effective in the prevention and treatment of biofilms, while also being harmless to human tissues.

In fact, the inventors of the present invention have found that vitamin E phosphate shows greater effectiveness against biofilms compared to other vitamins in the vitamin E group and particularly compared to vitamin E acetate. Moreover, it has surprisingly been found that when the vitamins in the vitamin E group are used in combination with methylene blue, anti-biofilm effectiveness proves to be boosted compared to the effectiveness of vitamin E per se, such as vitamin E acetate for example, owing to a synergistic effect between the vitamin and methylene blue.

The effectiveness of the vitamins according to the invention is observable against bacterial biofilms (of pathogenic gram-positive and gram-negative bacteria) and against fungal biofilms.

Vitamin E phosphate, or α-tocopherol phosphate, is a water-soluble form of α-tocopherol recently found in small amounts in the tissues and plasma of humans and other animals (Ogru E, Gianello R, Libinaki R, Smallridge A, Bak R, et al. (2003) Medimond Med Pub, Englewood N.J., USA 127-132. Vitamin E Phosphate: An endogenous form of vitamin E). Given that in α-tocopherol phosphate, the OH group responsible for antioxidant activity is phosphorylated, this molecule should have no antioxidant activity per se. However, Rezk et al. have proposed that this molecule acts as pro-vitamin E, maintaining strong antioxidant activity and showing some new regulating activities in the cells (Rezk B M, Haenen G R M M, van der Vijgh W J F, Bast A (2004). Biochim Biophys Acta 1683: 16-21. The extraordinary antioxidant activity of vitamin E phosphate). However, no studies have been conducted on the activity of α-tocopherol phosphate on bacteria yet.

According to the present invention, vitamin E phosphate, optionally combined with methylene blue, and/or vitamin E, such as vitamin E acetate for example, combined with methylene blue, can also be used in combination with a suitable biomaterial that can perform the function of a vitamin E carrier in the site of interest.

The use of various vectors for the release of drugs (“drug delivery system”) is known, including for example vectors made of polymethyl methacrylate (PMMA) or polyethylene glycol (PE) that are loaded with a drug, or vectors made of resorbable biomaterials such as hyaluronic acid and derivatives thereof, and polylactic acid.

The term hyaluronic acid (also indicated as HA herein below) is a generic term indicating polysaccharides derived from the polymerization of a repeating unit comprising D-glucuronic acid and N-acetyl-D-glucosamine. In the forms naturally present in many animal tissues, HA may have a molecular weight (PM) in the range of about 5,000 to about 20 million daltons (Da) and the properties thereof can vary according to its actual molecular weight. HA is a fundamental component of the extracellular matrix (ECM) and it is essential for good functioning of numerous body tissues, such as connective or epithelial tissues, inner ear fluids, the vitreous humour of the eyes and also joint fluid (synovia). It is a highly biocompatible and biodegradable polymer with well-known anti-adhesive and lubricating properties as described for example in the international patent application WO 2004/014303.

The grafting of biocompatible polyesters onto the N-acetyl-D-glucosamine moieties of HA, through the reaction with the free hydroxyl group present in these moieties, has also been studied. The HA derivative maintains high hydrophilicity, combined with the properties of viscosity and retention of the viscosity over time. The main component of the hydrogel that is obtained is a hyaluronic acid derivative, obtained by grafting chains of biodegradable and biocompatible polyesters on a fraction of the hyaluronic acid having a molecular weight within a given range. The other reactant in the production of the derivative is a biodegradable and biocompatible polyester or a mixture of polyesters or copolymers thereof. The most interesting polyesters are polylactic acid, polyglycolic acid, polycaprolactone, and mixtures and copolymers thereof.

Polylactic acid (also indicated as PLA herein below) is one of the most well-known biodegradable polyesters. This compound has been widely studied for use in tissue engineering and for drug delivery systems and it has also been widely used in the medical field. The use of PLA is preferred because of its excellent properties such as its mechanical, thermal and barrier properties, and its processability using traditional processing technologies such as extrusion, injection moulding, compression and blow moulding. PLA degradation has been studied in animal models and in humans for medical applications such as the use of prostheses, surgical sutures and PLA-based drug delivery systems. In the environment of the animal or human organism, PLA is initially degraded by hydrolysis and the soluble oligomers formed are metabolized by the cells. The applications of PLA in the medical field are due to its biocompatibility and to its solubility in the body by simple hydrolysis, which produces non-harmful, non-toxic degradation compounds.

Therefore, a specific object of the present invention is constituted by vitamin E for use in the treatment and prevention of biofilm infections or as an antifungal agent, wherein the vitamin E is selected from the group consisting in vitamin E phosphate, optionally in combination with methylene blue, and/or vitamin E, such as vitamin E acetate, in combination with methylene blue.

The vitamin E according to the present invention can be used against a gram-negative bacterial biofilm, such as for example biofilms formed by Escherichia coli and Pseudomonas aeruginosa, a gram-positive bacterial biofilm such as for example biofilms formed by Staphylococcus aureus and Staphylococcus epidermidis, or a fungal biofilm such as for example a biofilm formed by Candida albicans.

Therefore, the vitamin E according to the present invention can be employed for the prevention of infections by bacteria and fungi capable of forming biofilms, or for treatment against in situ bacteria and fungi capable of forming biofilms, but which have not yet formed the biofilm, or for treatment of an already formed biofilm. The vitamin E according to the present invention can also be employed as an antifungal agent regardless of the ability of the fungus to form a biofilm or the formation of the biofilm itself.

According to the present invention, vitamin E phosphate and/or vitamin E acetate can be used also at high concentrations of up to 100 w/v %, preferably at a concentration varying from 1 to 50 w/v %, and even more preferably from 5 to 20 w/v %.

The vitamin E according to the present invention can be applied on inert surfaces, such as on the surfaces of prostheses or implantable biomaterials (including by non-limiting example joint replacements, dental implants, breast implants, urological, cardiovascular, maxillofacial and ear, nose and throat prostheses, etc.), of material for osteosynthesis and fracture fixation, of catheters or endovascular devices, bone cements, surgical instruments, or living surfaces such as surfaces of wounds, of the skin, joints, bones, of internal tissues, soft tissues, and surgical areas for example.

Vitamin E phosphate per se, optionally in combination with methylene blue, and/or vitamin E, such as vitamin E acetate for example, in combination with methylene blue can be applied as such on surfaces or formulated in a pharmaceutical composition in combination with suitable excipients and/or adjuvants.

A further object of the present invention is constituted by a pharmaceutical composition comprising or consisting in vitamin E, as the active ingredient, and possibly pharmaceutically acceptable excipients and/or adjuvants, for use in the treatment and prevention of biofilm infections or as an antifungal agent. The pharmaceutical composition according to the present invention can further comprise a biomaterial.

According to the present invention, methylene blue has an antimicrobial and staining function serving to view the application in situ. Methylene blue can be used at concentrations ranging from 0.01 to 99 w/v %, preferably at concentrations ranging from 0.5 to 5 w/v %.

Among the biomaterials that can be used according to the present invention, the following hyaluronic acid-based compounds can be cited as examples: hyaluronic acid in its cross-linked or non-cross-linked form, salts or derivatives thereof, polylactic acid, polycaprolactone or their copolymers, polyethylene glycol, polymethyl methacrylate, or the biomaterial is a mixture of the compounds listed above. Among the derivatives of hyaluronic acid, the following can be mentioned: derivatives in which N-acetyl-D-glucosamine groups of hyaluronic acid are bound to a polyester of polylactic acid, polyglycolic acid, polycaprolactone or their copolymers, or with mixtures of said polyesters.

The molecular weight of the hyaluronic acid preferably ranges from 0.5 to 4 MDa.

Owing to its rheological and mechanical properties, the biomaterial can be applied to the inert or living surfaces cited above and release vitamin E locally and gradually as the biomaterial decomposes. In this manner, the vitamin E is able to carry out its action against biofilm infections, for example in a surgical site.

The composition according to the present invention can contain vitamin E phosphate and/or vitamin E such as vitamin E acetate up to concentrations of 100 w/v %, preferably at a concentration ranging from 1 to 50 w/v % and even more preferably from 5 to 20 w/v %, in which said percentage is the percentage of vitamin E by weight with respect to the volume of the total composition.

As stated above, the biofilm that can be treated or inhibited in formation is a gram-negative bacterial biofilm, such as for example biofilms formed by Escherichia coli and Pseudomonas aeruginosa, a gram-positive bacterial biofilm, such as for example biofilms formed by Staphylococcus aureus and Staphylococcus epidermidis, or a fungal biofilm such as for example a biofilm formed by Candida albicans. The composition of the invention can also be employed as an antifungal agent regardless of the ability of the fungus to form a biofilm or the formation of the biofilm itself.

The pharmaceutical composition of the present invention can be applied on inert surfaces, such as the surfaces of prostheses or implantable biomaterials (including by non-limiting example joint replacements, dental implants, breast implants, urological, cardiovascular, maxillofacial and ear, nose and throat prostheses, etc.), of material for osteosynthesis and fracture fixation, of catheters or endovascular devices, bone cements, surgical instruments, or on living surfaces such as surfaces of wounds, of the skin, joints, bones, and of internal tissues for example.

The pharmaceutical composition according to the present invention thus represents a preparation that is ready for use, wherein the biomaterial, if present, is preloaded with predetermined doses of vitamin E. The composition is therefore ready to be applied in the surgical areas or on the surface of prostheses to be implanted.

The present invention therefore concerns a kit for the prevention or treatment of biofilm infections or as an antifungal agent, said kit comprising the pharmaceutical composition as defined above and an applicator means for applying said composition. The composition can be contained in a syringe or in a vial for topical application. The applicator means can consist of a cannula and/or a means for applying the formulation in spray form.

According to an alternative embodiment, the present invention concerns a kit for use in the prevention or treatment of biofilm infections or as an antifungal agent, said kit comprising or consisting in vitamin E and a biomaterial, each separated from the other. Therefore, the kit makes it possible to mix vitamin E and the biomaterial at the desired ratio based on the needs of the case at hand shortly before application in the surgical area or on the surface of the prosthesis to be implanted. The vitamin E can be vitamin E phosphate, optionally in combination with methylene blue, and/or vitamin E, such as vitamin E acetate for example, in combination with methylene blue.

The kit can further comprise a means for mixing the vitamin E and the biomaterial, and possibly an applicator means for applying the vitamin E mixed with the biomaterial.

The kit could be constituted by vials and/or syringes containing the vitamin E, the excipients and/or adjuvants and the biomaterial possibly to be reconstituted, and by a mixing system using a spatula or in a syringe or using dedicated accessories to obtain the vitamin E and the adjuvant and/or excipient included in the biomaterial. The vitamin E and the biomaterial can be contained in syringes or vials for topical application. The applicator means can consist of a cannula and/or a means for applying the formulation in spray form.

As stated above, among the biomaterials that can be used according to the present invention, the following hyaluronic acid-based compounds can be cited as examples: hyaluronic acid in its cross-linked or non-cross-linked form, salts or derivatives thereof, polylactic acid, polycaprolactone or their copolymers, polyethylene glycol, polymethyl methacrylate, or the biomaterial is a mixture of the compounds listed above. Among the derivatives of hyaluronic acid, the following can be mentioned: derivatives in which N-acetyl-D-glucosamine groups of hyaluronic acid are bound to a polyester of polylactic acid, polyglycolic acid, polycaprolactone or their copolymers, or with mixtures of said polyesters.

The molecular weight of the hyaluronic acid preferably ranges from 0.5 to 4 MDa.

The invention is described herein below by way of non-limiting example, with particular reference to several illustrative examples and to the attached drawings, of which:

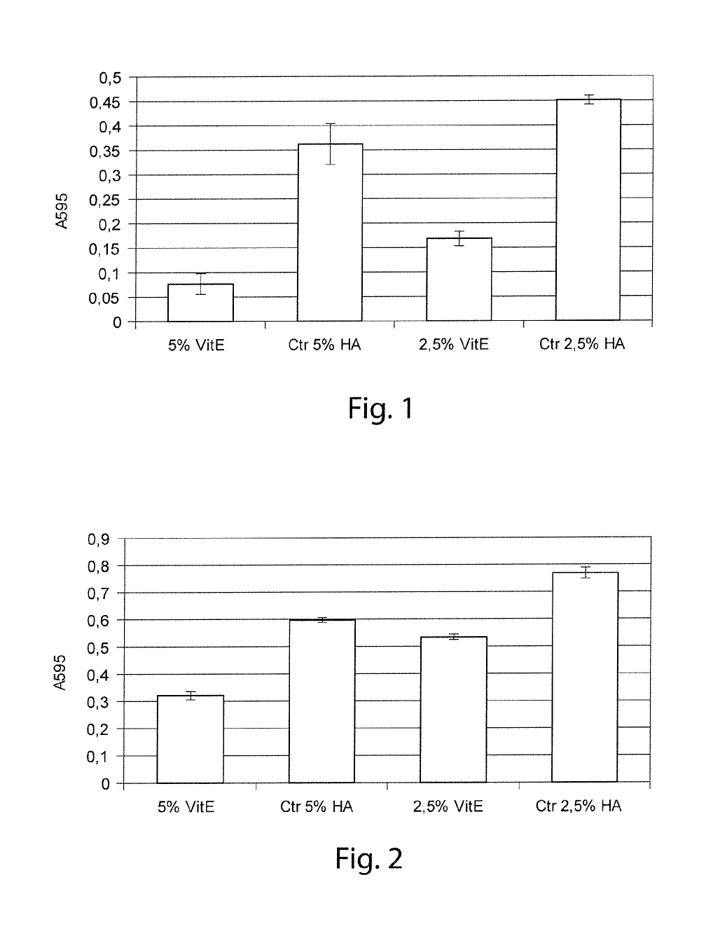

FIG. 1 shows the results of the semi-quantitative analysis of the microbial biofilm produced by S. aureus by means of a spectrophotometric assay. The data are expressed as means±SD. P<0.05.

FIG. 2 shows the results of the semi-quantitative analysis of the microbial biofilm produced by S. epidermidis by means of a spectrophotometric assay. The data are expressed as means±SD. P<0.05.

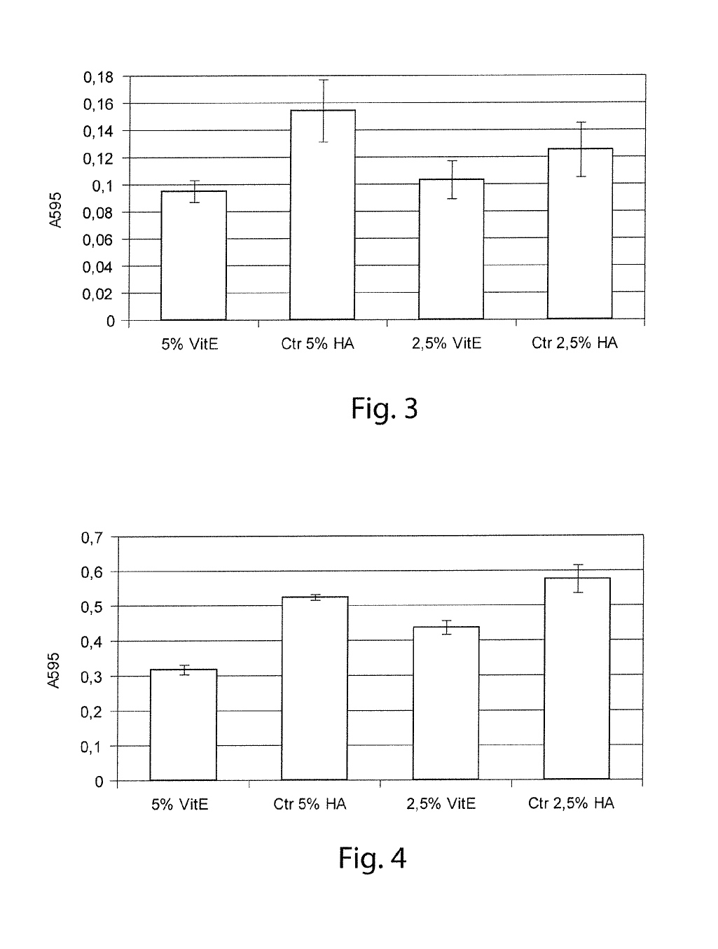

FIG. 3 shows the results of the semi-quantitative analysis of the microbial biofilm produced by E. coli by means of a spectrophotometric assay. The data are expressed as means±SD. P<0.05.

FIG. 4 shows the results of the semi-quantitative analysis of the microbial biofilm produced by P. aeruginosa by means of a spectrophotometric assay. The data are expressed as means±SD. P<0.05.

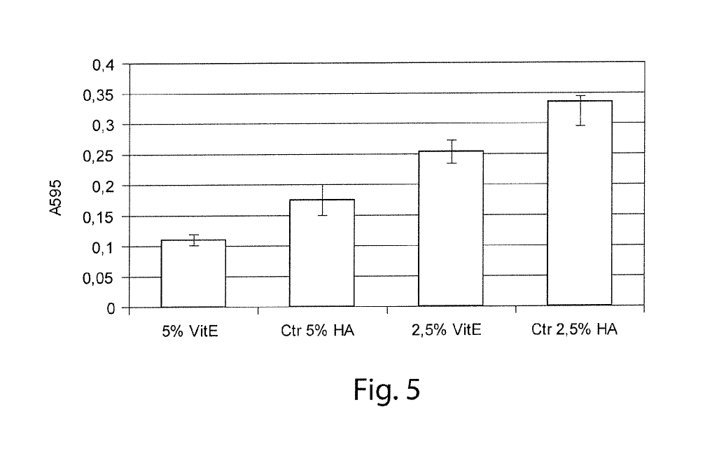

FIG. 5 shows the results of the semi-quantitative analysis of the microbial biofilm produced by C. albicans by means of a spectrophotometric assay. The data are expressed as means±SD. P<0.05.

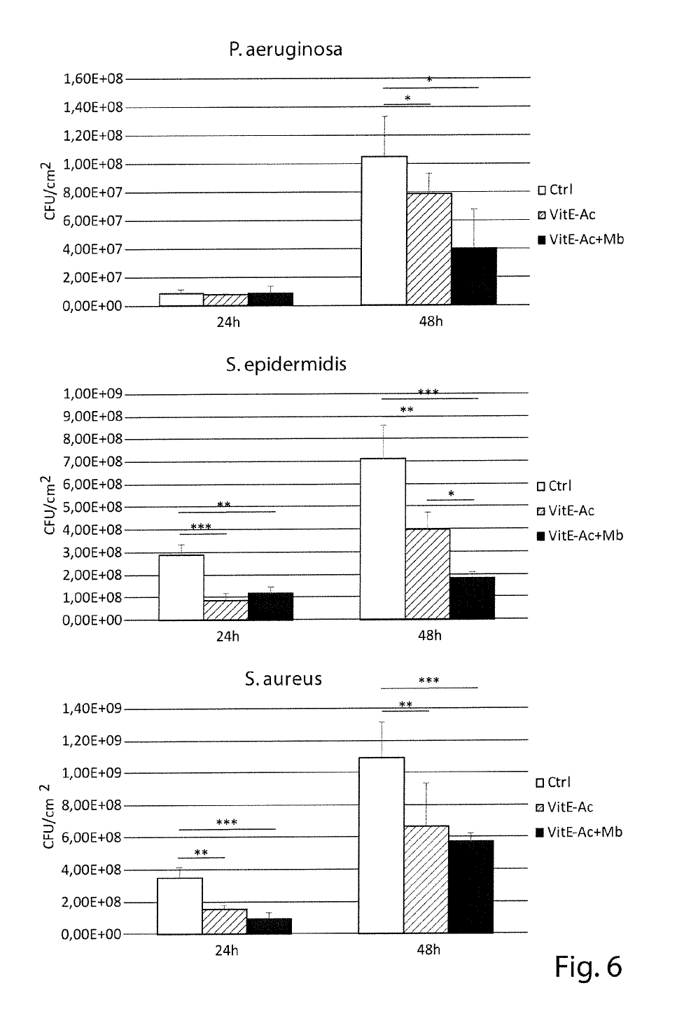

FIG. 6 shows the anti-biofilm activity of vitamin E acetate and of vitamin E acetate in combination with methylene blue. VitE-Ac=Vitamin E acetate; Mb=methylene blue; * P≤0.05, ** P≤0.001, *** P≤0.0001.

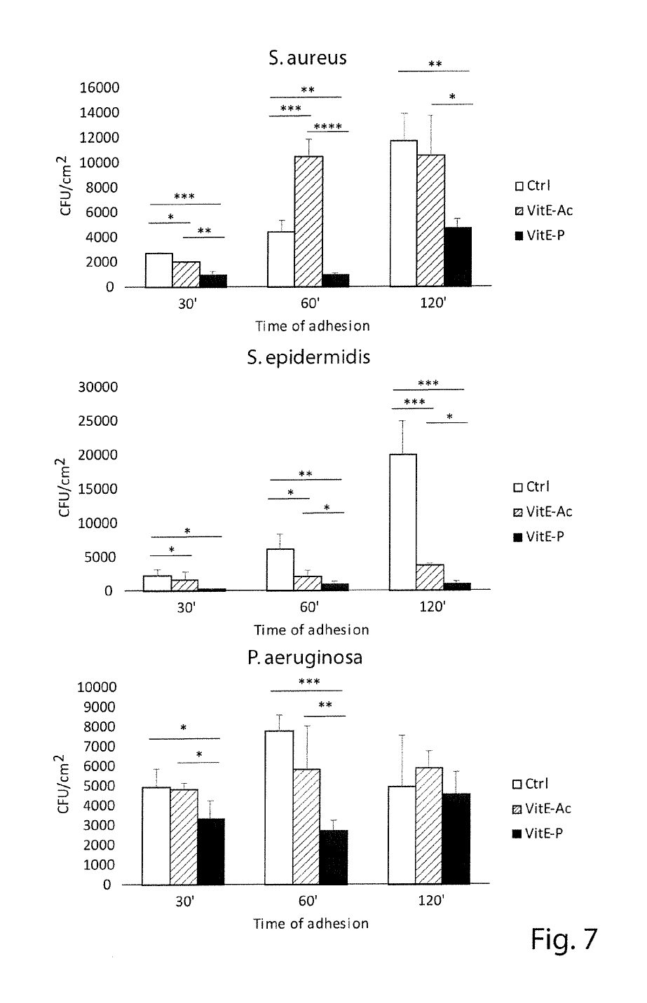

FIG. 7 shows the anti-adhesion activity of vitamin E acetate and vitamin E phosphate: VitE-Ac=Vitamin E acetate (5 mg/cm²); VitE-P=Vitamin E phosphate (5 mg/cm²).

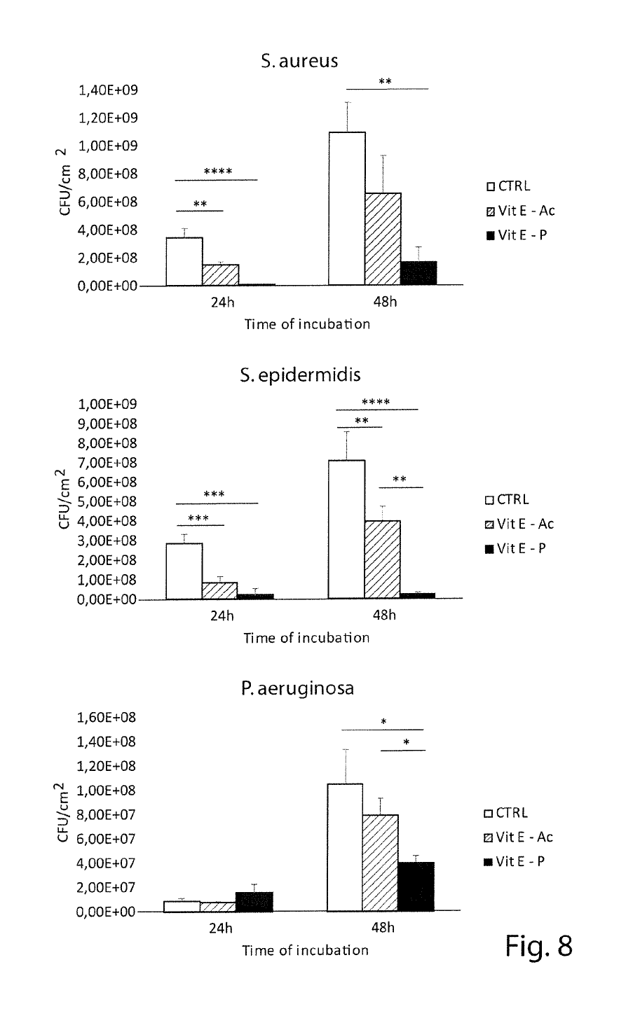

FIG. 8 shows the anti-biofilm activity of vitamin E acetate and vitamin E phosphate: VitE-Ac=Vitamin E acetate (5 mg/cm²); VitE-P=Vitamin E phosphate (5 mg/cm²).

### EXAMPLE 1: IN VITRO STUDY OF THE ANTI-BIOFILM ACTIVITY OF VITAMIN E AGAINST GRAM-NEGATIVE AND GRAM-POSITIVE BACTERIA AND FUNGI

Materials and Methods

Vitamin E (VITAMIN ACETATE purchased from SIGMA ALDRICH) was diluted at various concentrations in hyaluronic acid utilized as the carrier gel. Its anti-biofilm activity was tested in vitro against gram-positive strains (Staphylococcus aureus, Staphylococcus epidermidis), gram-negative strains (Escherichia coli, Pseudomonas aeruginosa) and fungi (Candida albicans).

Microbial Strains

The microbial strains utilized in this study were isolated from clinical samples collected from bone and soft tissue infections. In particular, strains of S. aureus, S. epidermidis, E. coli, P. aeruginosa and C. albicans were utilized. They were identified beforehand by means of biochemical assays and examined to assess their ability to produce biofilms, according to the spectrometric assay described by Christensen et al. (1985). The microorganisms were stored at −80° C. prior to the analysis. Prior to use, the strains were thawed and cultivated in an appropriate culture medium at 37° C. for 24 h.

Assessment of Antimicrobial Activity

Antimicrobial activity was assessed by determining the minimum inhibitory concentration (MIC). MIC, defined as the lowest concentration of an antimicrobial substance that inhibits the visible growth of a microorganism, was determined using the broth microdilution method, in accordance with the guidelines of the European Committee on Antimicrobial Susceptibility Testing (EUCAST).

In short, a microbial suspension was prepared at an optical density of 0.5 McFarland in a brain-heart infusion broth (BHI, bioMérieux SA, Marcy l'Etoile, France). After obtaining a microbial load of 1×10⁵ CFU/ml using suitable dilutions, 20 μl of microbial suspension were inoculated in a cell culture plate with 96 wells containing 180 μl of a 1:2 serial dilution in BHI of a starting solution of Vitamin E (VitE) at a concentration of 50 mg/ml. VitE was diluted in hyaluronic acid (HA) at various concentrations, for which the growth checks were performed by inoculating the microbial suspension in BHI+HA. The MIC values were read after 24 hours of incubation at 37° C. The assay was conducted in duplicate and repeated twice.

Assessment of Anti-Biofilm Activity

The bacterial biofilm was formed in a sterile 96-well polystyrene plate. In short, a microbial suspension was prepared at an optical density of 0.5 McFarland for each strain. 20 μl of microbial suspension were added to each well containing 180 μl of VitE at various concentrations. Positive controls were performed by inoculating the bacteria in BHI+HA. Following incubation for 24 h at 37° C., the broth was removed, together with non-adherent microorganisms, and 200 μl of broth were added once again. The plates were incubated at 37° C. for another 48 h, until a mature biofilm was obtained.

Semi-Quantitative Analysis of the Biofilm by Means of a Spectrophotometric Test

The amount of biofilm was determined by means of a spectrophotometric assay, adopting the method developed by Christensen et al. (1985). At the end of the incubation period, the culture medium was removed and the wells covered by the biofilm were washed twice with a sterile saline solution and then dried. The biofilm was stained with 200 μl of a 5% crystal violet solution for 10 minutes. After staining, the wells were washed with a sterile saline solution to remove excess stain and left to dry completely. Lastly, 200 μl of 96% ethanol were added to each well to re-solubilize the stain included in the biofilm. Absorbance of the crystal violet was measured at 595 nm using a microplate reader. The assay was conducted in triplicate for each bacterial strain and repeated twice.

Statistical Analyses

The results are expressed as means±standard deviations (SD). Comparisons between two groups were carried out using Student's t-test. A value of P<=0.05 was used as the level of significance.

Results

Assessment of Antimicrobial Activity

Vitamin E showed inhibitory activity against all the strains tested, with a mean MIC value of 2.5%.

Assessment of Anti-Biofilm Activity Against S. aureus

The results are reported in FIGS. 1-5.

The concentration of 5% VitE alone significantly reduced microbial biofilm development for all the strains tested. With respect to the control, the reduction percentages were equal to 79% for S. aureus, 46% for S. epidermidis, 38% for E. coli, 39% for P. aeruginosa, and 37% for C. albicans.

The concentration of 2.5% VitE also significantly reduced the microbial biofilm. With respect to the control, the percentages of reduction observed were equal to 63% for S. aureus, 30% for S. epidermidis, 18% for E. coli and 24% for P. aeruginosa and C. albicans.

Higher concentrations of vitamin E can have a greater effect against the formation of biofilms produced by gram-positive and gram-negative bacteria and fungi.

The results concerning the antibacterial and anti-biofilm activity of the same concentrations of VitE (i.e., 2.5% and 5%) used in a formulation without the use of the HA carrier were comparable to those reported above.

### EXAMPLE 2: STUDY OF THE ANTI-BIOFILM ACTIVITY OF VITAMIN E ACETATE AND OF VITAMIN E ACETATE IN COMBINATION WITH METHYLENE BLUE

Materials and Methods

Reactants

The following reactants were used: vitamin E acetate (α-tocopherol acetate, Alfa Aesar, Heysham, United Kingdom) and methylene blue (Merk, Darmstadt, Germany). The solutions were prepared by dissolving vitamin E acetate in ethanol (250 mg/ml) and methylene blue in distilled water (50 mg/ml).

Bacterial Strains

This study used clinically relevant strains isolated from patients with prosthetic joint infections at the Microbiology Laboratory of the IRCCS Galeazzi Orthopaedic Institute (Italy). In particular, the following were selected: a strain of methicillin-resistant Staphylococcus epidermidis, a strain of methicillin-resistant Staphylococcus aureus and a strain of Pseudomonas aeruginosa. These strains were identified beforehand by means of biochemical assays (VITEK® 2 Compact, bioMérieux, Marcy l'Etoile, France) and stored at −80° C. prior to the analysis. Before beginning the study, all strains underwent testing to assess their ability to produce biofilms according to the spectrophotometric assay described by Christensen et al. (J Clin Microbiol 1985; 22: 996-1006).

Disk Coating and Biofilm Formation

Sterile disks of sandblasted titanium with a diameter of 25 mm and a thickness of 5 mm (Adler Ortho, Cormano, Italy) were used as the substrate for the formation of biofilms. The disks were coated with sub-inhibitory concentrations of vitamin E acetate (5 mg/cm²) or vitamin E acetate with the addition of methylene blue (20 μg/cm²). The concentrations were selected based on the minimum inhibitory concentrations (data not shown). Uncoated disks were used as negative controls. Subsequently, an overnight bacteria culture was resuspended in Brain Heart Infusion Broth (BHI; Biomérieux) at a density of 1.5×10⁸ CFU/ml for each strain, and 200 microliters of microbial suspension were inoculated in sterile polystyrene plates with 6 wells containing coated and uncoated disks and 4.8 ml of BHI. The plates were incubated at 37° C. in an aerobic atmosphere for 24 and 48 hours. The test was performed in triplicate for each strain.

Detachment of the Biofilm and CFU Count

Upon completion of the biofilm incubation time, the disks were washed twice with a sterile saline solution to eliminate non-adherent bacteria. The disks were then immersed in 5 ml of a 0.1 w/v % dithiothreitol solution (DTT, Sigma-Aldrich, Milan, Italy) and mechanically stirred for 15 minutes at room temperature. Suitable dilutions of the fluids obtained were inoculated on Tryptic Soy Agar (TSA, Merck) and incubated at 37° C. in an aerobic atmosphere for 24 hours for the CFU count.

Statistical Analysis

The results are expressed as means±standard deviations (SD). Comparisons were made using two-way ANOVA followed by Tukey's post-hoc test. A P value equal to or lower than 0.05 was used as the level of significance.

Results

The results are represented in FIG. 6. Both vitamin E acetate and vitamin E+methylene blue revealed inhibitory activity against the biofilm produced by S. epidermidis and S. aureus on titanium disks. Reductions with respect to the controls were significant after 24 hours and after 48 hours for S. aureus and S. epidermidis. In the case of P. aeruginosa, the two formulations were effective after 48 hours, but not after 24 hours. The addition of methylene blue produced a slight increase in the activity of vitamin E acetate for all strains, especially after 48 hours of incubation, although the addition of methylene blue led to a significant reduction only in the case of S. epidermidis.

Conclusions

The results of this study revealed an anti-biofilm activity of vitamin E acetate that markedly increased after the addition of methylene blue.

### EXAMPLE 3: THE STUDY OF THE ANTI-BIOFILM ACTIVITY OF VITAMIN E ACETATE AND VITAMIN E PHOSPHATE

Materials and Methods

Reactants

In this study, the following reactants were used: vitamin E acetate (α-tocopherol acetate, Alfa Aesar, Heysham, United Kingdom) and vitamin E phosphate (α-tocopherol phosphate disodium salt; Sigma-Aldrich, St. Louis, Mo., USA). The solutions were prepared by dissolving vitamin E acetate in ethanol (250 mg/ml) and vitamin E phosphate in distilled water (250 mg/ml).

Bacterial Strains

This study used clinically relevant strains isolated from patients with prosthetic joint infections at the Microbiology Laboratory of the IRCCS Galeazzi Orthopaedic institute (Italy). In particular, the following were selected: strains of methicillin-resistant Staphylococcus epidermidis, of methicillin-resistant Staphylococcus aureus and of Pseudomonas aeruginosa. These strains were identified beforehand by means of biochemical assays (VITEK® 2 Compact, bioMérieux, Marcy l'Etoile, France) and stored at −80° C. prior to the analysis. Before beginning the study, all strains were screened to assess their ability to produce biofilms, according to the spectrophotometric assay described by Christensen et al. (J Clin Microbiol 1985; 22: 996-1006).

Assessment of Antimicrobial Activity

The bacteriostatic and bactericidal activity of vitamin E acetate and vitamin E phosphate against the above-mentioned bacterial strains was assessed by measuring the minimum inhibitory concentration (MIC) and the minimum bactericidal concentration (MBC). MIC, defined as the lowest concentration capable of inhibiting bacterial growth, was determined using the broth microdilution method according to EUCAST guidelines (http://www.eucast.org/clinical_breakpoints/).

In short, a suspension in Brain Heart Infusion Broth (BHI; Biomérieux, Marci l'Etoile, France) was prepared for each bacterial strain, with an optical density of 0.5 McFarland (1.5×10⁸ CFU/ml). The P. acnes broth was supplemented with 5% defibrinated sheep blood (Liofilchem, Roseto degli Abruzzi, Italy). After a concentration of 10⁵ UFC/ml was obtained using appropriate dilutions, each suspension was inoculated in a 96-well microtitre plate containing a two-fold serial dilution of vitamin E acetate (VitE-Ac) and vitamin E phosphate. The controls were performed by inoculating bacterial suspensions in BHI alone. MIC values, corresponding to the lowest concentration showing no visible bacterial growth, were read after 24 hours of incubation at adequate conditions (except for P. acnes, which was incubated for 48 hours). MBC was determined by plating on agar plates 10 μl from each well that showed no turbidity. After incubation at adequate conditions, MBC was read as the lowest concentration capable of killing 99.9% of the initial inoculum.

Disk Coating and Assessment of Anti-Adhesion Activity

Sterile disks of sandblasted titanium with a diameter of 25 mm and a thickness of 5 mm (Adler Ortho, Cormano, Italy) were used as the substrate for the formation of biofilms. The disks were coated with sub-inhibitory concentrations of vitamin E acetate (5 mg/cm²) or vitamin E phosphate (5 mg/cm²). The concentrations were selected based on the MIC results. Uncoated disks were used as negative controls. The anti-adhesion activity of vitamin E was assessed against a representative strain for each species. An overnight bacteria culture was resuspended in Brain Heart Infusion Broth (BHI; Biomérieux) at a density of 1.0×10⁷ CFU/ml for each strain, and 400 microliters of microbial suspension were inoculated on coated and uncoated disks. The test was performed in triplicate for each strain. After 30, 60 and 120 minutes of incubation, the disks were washed twice with a sterile saline solution to eliminate non-adherent bacteria. The disks were then immersed in 5 ml of a 0.1 w/v % dithiothreitol solution (DTT, Sigma-Aldrich, Milan, Italy) and mechanically stirred for 15 minutes at room temperature. Suitable dilutions of the fluids obtained were inoculated on Tryptic Soy Agar (TSA, Merck) and incubated at 37° C. in an aerobic atmosphere for 24 hours for the CFU count.

Biofilm Formation

Coated and uncoated titanium disks were prepared as described above and utilized as the substrate for the formation of biofilms. Subsequently, an overnight bacteria culture was resuspended in Brain Heart Infusion Broth (BHI; Biomérieux) at a density of 1.5×10⁸ CFU/ml for each strain, and 200 microliters of microbial suspension were inoculated in sterile polystyrene plates with 6 wells containing coated and uncoated disks and 4.8 ml of BHI. The plates were incubated at 37° C. in an aerobic atmosphere for 24 and 48 hours. The test was performed in triplicate for each strain.

At the end of the biofilm incubation time, the disks were treated as described above and the detached cells of the biofilm were inoculated on Tryptic Soy Agar and incubated at 37° C. in an aerobic atmosphere for 24 hours for the CFU count.

Statistical Analysis

The results are expressed as means±standard deviations (SD). Comparisons were made using two-way ANOVA followed by Tukey's post-hoc test. A P value equal to or lower than 0.05 was used as the level of significance.

Results

Assessment of Antimicrobial Activity

As can be seen from Table 1, which reports the MIC and MBC values for vitamin E phosphate and vitamin E acetate, only vitamin E phosphate revealed antimicrobial activity against the strains tested, whereas the acetate form of the vitamin was ineffective at the concentrations tested (the maximum concentration utilized for both formulations was 20 mg/ml, due to solubility limits). P. acnes appeared to be the microorganism which was most sensitive to vitamin E phosphate, with MIC values ranging from 1.56 to 6.25 mg/ml. The MIC values for S. epidermis and S. aureus were more variable (3.13-100 mg/ml and 25-100 mg/ml, respectively). P. aeruginosa was the least susceptible strain, with MIC values varying between 100 and 200 mg/ml. At the concentrations tested, vitamin E phosphate proved to be bactericidal against P. acnes, S. epidermidis and S. aureus, but not against P. aeruginosa (see MBC values, Table 1).

TABLE 1

| | Vitamin E phosphate | | Vitamin E acetate | |
|---|---|---|---|---|
| | MIC | MBC | MIC | MBC |
| | (mg/ml) | (mg/ml) | (mg/ml) | (mg/ml) |
| Microorganism | range | range | range | range |
| S. aureus | 25-100 | 25-200 | >200 | >200 |
| S. epidermidis | 3.13-100 | 25-100 | >200 | >200 |
| P. aeruginosa | 100-200 | >200 | >200 | >200 |
| P. acnes | 1.56-6.25 | 31.25-50 | >200 | >200 |

Assessment of Anti-Adhesion and Anti-Biofilm Activity

The results concerning the anti-adhesion activity of the two compounds tested are represented in FIG. 7.

The effect of vitamin E phosphate was a significant decrease for all the strains tested, with the exception of the last time period tested for P. aeruginosa. The greatest effects were observed in the case of S. epidermidis, in which adhesion was almost completely inhibited (84-95%, with respect to the control), and in the case of S. aureus (60-78%, with respect to the control). A more moderate effect was observed in the case of P. aeruginosa (7-65%).

Vitamin E acetate caused a significant decrease in adhesion at all time periods in the case of S. epidermidis and S. aureus. No effect was observed in the case of P. aeruginosa.

The anti-biofilm activity of vitamin E acetate and vitamin E phosphate is illustrated in FIG. 8.

Both compounds revealed an inhibitory effect on the biofilm formed by S. epidermidis and S. aureus and P. aeruginosa on the titanium disks. Reductions with respect to the controls were significant after 24 hours and after 48 hours for S. epidermidis. In the case of S. aureus, adhesion was significantly inhibited by both compounds after 24 hours and only by vitamin E phosphate after 48 hours.

In the case of P. aeruginosa, no effects were observed at the earlier time periods, but both formulations showed a significant effect after 48 hours.

### CONCLUSIONS

The results of these tests demonstrated that vitamin E phosphate has stronger and more significant anti-adhesion and anti-biofilm activity than vitamin E acetate, in the case of S. epidermidis and S. aureus. Moreover, the results demonstrated that only vitamin E phosphate displays activity against P. aeruginosa. Furthermore, vitamin E phosphate revealed antimicrobial activity, which was not observed with vitamin E acetate. The fact that an effect of vitamin E acetate is lacking is probably due to the high hydrophobicity of this vitamin.

1-18. (canceled)

19. Vitamin E for use in the treatment and prevention of biofilm infections, such as bacterial or fungal biofilm, wherein the vitamin E is selected from the group consisting of vitamin E phosphate, optionally in combination with methylene blue, and/or vitamin E, such as vitamin E acetate, in combination with methylene blue.

20. Vitamin E according to claim 19, wherein the biofilm is a gram-negative bacterial biofilm, such as biofilms formed by Escherichia coli and Pseudomonas aeruginosa, a gram-positive bacterial biofilm containing bacteria such as biofilms formed by Staphylococcus aureus and Staphylococcus epidermidis, or a fungal biofilm such as a biofilm formed by Candida albicans for example.

21. Vitamin E according to claim 19, wherein vitamin E phosphate and/or vitamin E such as vitamin E acetate for example is used at a concentration of up to 100 w/v %, preferably ranging from 1 to 50 w/v % and even more preferably from 5 to 20 w/v %.

22. Vitamin E according to claim 19, wherein the vitamin E is applied on living surfaces such as surfaces of wounds, of the skin, joints, bones, and of internal tissues for example.

23. Use of vitamin E as defined in claim 19 against the formation of a biofilm on inert surfaces by applying said vitamin E on to the inert surfaces, such as on the surfaces of prostheses or implantable biomaterials, of material for osteosynthesis and fracture fixation, of catheters or endovascular devices, bone cements, surgical instruments.

24. Pharmaceutical composition comprising or consisting of vitamin E, as the active ingredient, and possibly pharmaceutically acceptable excipients and/or adjuvants, for use in the treatment and prevention of biofilm infections, such as bacterial or fungal biofilm, wherein the vitamin E is selected from the group consisting of vitamin E phosphate, optionally in combination with methylene blue, and/or vitamin E, such as vitamin E acetate, in combination with methylene blue.

25. Pharmaceutical composition according to claim 24, further comprising a biomaterial.

26. Pharmaceutical composition according to claim 25, wherein the biomaterial is selected from the group consisting of hyaluronic acid-based compounds such as for example hyaluronic acid, in its cross-linked or non-cross-linked form, salts or derivatives thereof, such as derivatives in which N-acetyl-D-glucosamine groups of hyaluronic acid are bound to a polyester of polylactic acid, of polyglycolic acid, of polycaprolactone or of their copolymers, or with mixtures of said polyesters, polylactic acid, polycaprolactone or their copolymers, polyethylene glycol, polymethyl methacrylate, or the biomaterial is a mixture of the compounds listed above.

27. Pharmaceutical composition according to claim 26, wherein the hyaluronic acid has a molecular weight ranging from 0.5 to 4 MDa.

28. Pharmaceutical composition according to claim 24, wherein vitamin E phosphate and/or vitamin E, such as vitamin E acetate for example, is at a concentration of 1 to 50 w/v %, preferably 5 to 20 w/v %.

29. Pharmaceutical composition according to claim 24, wherein the biofilm is a gram-negative bacterial biofilm, such as biofilms formed by Escherichia coli and Pseudomonas aeruginosa, a gram-positive bacterial biofilm such as biofilms formed by Staphylococcus aureus and Staphylococcus epidermidis, or a fungal biofilm such as a biofilm formed by Candida albicans for example.

30. Pharmaceutical composition according to claim 24, wherein said composition is applied on living surfaces such as surfaces of wounds, of the skin, joints, bones, and of internal tissues for example.

31. Use of the pharmaceutical composition as defined in claim 24 against the formation of a biofilm on inert surfaces by applying said pharmaceutical composition on to the inert surfaces, such as on the surfaces of prostheses or implantable biomaterials, of material for osteosynthesis and fracture fixation, of catheters or endovascular devices, of bone cements, of surgical instruments.

32. Kit for use in the prevention or treatment of biofilm infections, such as bacterial or fungal biofilm, said kit comprising or consisting of vitamin E and a biomaterial, separated from the other, wherein the vitamin E is selected from the group consisting of vitamin E phosphate, optionally in combination with methylene blue, and/or vitamin E such as vitamin E acetate in combination with methylene blue.

33. Kit according to claim 32, further comprising a means for mixing the vitamin E and the biomaterial.

34. Kit for use according to any one of claim 32, wherein said biomaterial is selected from the group consisting in hyaluronic acid-based compounds such as for example hyaluronic acid, in its cross-linked or non-cross-linked form, salts or derivatives thereof, such as derivatives wherein N-acetyl-D-glucosamine groups of hyaluronic acid are bound to a polyester of polylactic acid, of polyglycolic acid, of polycaprolactone or their copolymers, or with mixtures of said polyesters, polylactic acid, polycaprolactone or their copolymers, polyethylene glycol, polymethyl methacrylate, or the biomaterial is a mixture of the compounds listed above.

35. Kit for use according to claim 33, wherein the molecular weight of the hyaluronic acid ranges from 0.5 to 4 MDa.

36. Kit for use according to claim 32, said kit further comprising an applicator means for applying the vitamin E mixed with the biomaterial.

37. Kit for use in the prevention or treatment of biofilm infections, such as bacterial or fungal biofilm, said kit comprising or consisting of the pharmaceutical composition as defined in claim 24 and an applicator means for applying said composition.
